(19) United States
(12) Patent Application Publication
(10) Pub. No.: US 2018/0271758 A1
(43) Pub. Date: Sep. 27, 2018

(54) Cosmetic Antiperspirant Composition

(71) Applicant: Thomas Brunner Hygiene GmbH, Schlierbach, OT (DE)

(72) Inventor: Thomas Brunner, Jr., Schlierbach (DE)

(21) Appl. No.: 15/522,742

(22) PCT Filed: Oct. 30, 2015
(86) PCT No.: PCT/EP2015/075253

§ 371 Date: Apr. 27, 2017

(30) Foreign Application Priority Data
Oct. 31, 2014 (DE) 10 2014 222 270.2

(51) Int. Cl. A61K 8/365; A61K 8/368; A61Q 15/00

(52) U.S. Cl. CPC A61K 8/365; A61K 8/368; A61K 8/9728; A61K 2800/30; A61Q 15/00

(57) ABSTRACT

A cosmetic antiperspirant composition, i.e., a perspiration-inhibiting or sweat-inhibiting composition, which comprises at least one active antiperspirant ingredient selected from the group consisting of hydroxycarboxylic acid, hydroxycarboxylic salt, and mixtures thereof. Preferably, the cosmetic antiperspirant composition is free of salts selected from the group consisting of aluminum salts, zirconium salts, and mixtures thereof

This application is a United States National Stage Application claiming the benefit of priority under 35 U.S.C. 371 from International Patent Application No. PCT/EP2015/075253 filed Oct. 30, 2015, which claims the benefit of priority from German Patent Application Serial No. DE 102014222270.2 filed Oct. 31, 2014, the entire contents of which are herein incorporated by reference.

### FIELD OF USE AND PRIOR ART

The invention relates to a cosmetic antiperspirant composition, to a cosmetic product, and also to hydroxycarboxylic acids or salts thereof for use in the production of cosmetic antiperspirant compositions free of aluminum salt.

Antiperspirants generally are substances which reduce the activity of the sweat glands and also the amount of perspiration delivered and therefore indirectly reduce body odor.

The mode of action of commercial antiperspirant compositions is derived from the presence of aluminum salts, such as aluminum chlorohydrates, for example, as active antiperspirant ingredients. The activity of the aluminum salts here is astringent and denaturing. The astringent effect leads to a narrowing of the sweat ducts. On account of the denaturing effect, the sweat proteins are precipitated to form a protein plug on the outer end of the sweat ducts of the sweat glands, with the consequence overall that less perspiration is able to emerge.

Antiperspirant compositions of this kind are known, for example, from publications DE 10 2011 086 019 A1, DE 10 2007 063 352 A1, EP 2 481 392 A2, WO 2001/047476 A2, WO 2003/041674 A1, WO 2005/112879 A1, and WO 2006/066704 A1.

A disadvantage with the commercial antiperspirant compositions is that the aluminum salts they contain are suspected of causing permanent damage to the human body. Aluminum salts, for instance, are suspected of triggering breast cancer or Alzheimer's.

Although scientific evidence supporting beyond doubt detrimental effects on health of aluminum salt-containing cosmetics has not so far been furnished, there is a demand for antiperspirant compositions which do not encounter medical concerns.

Problem and Solution

The problem addressed with the present invention was therefore that of providing a cosmetic antiperspirant composition and also a cosmetic product which engage with the demand identified above and which avoid disadvantages known from the prior art.

This problem is solved by a cosmetic antiperspirant composition free of aluminum salt and comprising at least one active antiperspirant ingredient selected from the group consisting of hydroxycarboxylic acid, hydroxycarboxylic salt, and mixtures thereof and by a cosmetic product that contains to 100 wt % the following ingredients:
5 wt % to 30 wt % of the at least one active antiperspirant ingredient,
10 wt % to 50 wt % of ethanol,
0.5 wt % to 5 wt % of Saccharomyces ferment,
0 wt % to 2.5 wt % of at least one pH regulator,
0 wt % to 2.5 wt % of at least one complexing agent,
0 wt % to 2.0 wt % of at least one viscosity regulator and/or film-former,
0 wt % to 2 wt % of at least one surfactant,
0 wt % to 30.5 wt % of at least one skincare substance,
0 wt % to 0.2 wt % of at least one fragrance, and
10 wt % to 75 wt % of water.

According to a first aspect, the invention relates to a cosmetic antiperspirant composition, i.e., a perspiration-inhibiting or sweat-inhibiting composition, which comprises at least one active antiperspirant ingredient selected from the group consisting of hydroxycarboxylic acid, hydroxycarboxylic salt, and mixtures thereof.

Underlying the present invention is the surprising finding that hydroxycarboxylic acids and/or salts thereof, especially the hydroxycarboxylic acids and/or hydroxycarboxylic salts elucidated in more detail hereinafter, are suitable for substituting aluminum salts contained in commercial antiperspirant compositions.

The cosmetic antiperspirant composition advantageously reduces, in particular, the activity of apocrine sweat glands, which are located in particular in the region of the axilla (pyramid-shaped anatomical space between the lateral chest wall and the medial upper arm), the nipple, in the groin area, the perianal region (glandulae circumanales), and the pubic region (woman: mons pubis, labia majora; man: scrotum).

The term “hydroxycarboxylic acid” in the sense of the present invention may mean both one type of hydroxycarboxylic acid and two or more different types of hydroxycarboxylic acid. Correspondingly, the term “hydroxycarboxylic salt” in the sense of the present invention may mean one type of hydroxycarboxylic salt or two or more different types of hydroxycarboxylic salt.

The observations below concerning the hydroxycarboxylic acid also apply, insofar as is rational, to the hydroxycarboxylic salt.

In one preferred embodiment the cosmetic antiperspirant composition is free of salts selected from the group consisting of aluminum salts, zirconium salts, and mixtures thereof. In other words, it is preferred in accordance with the invention for the cosmetic antiperspirant composition to be aluminum and/or zirconium salt-free.

According to a further embodiment, the cosmetic antiperspirant composition is aluminum, zirconium, and optionally zinc salt-free.

With particular preference the antiperspirant composition is aluminum salt-free, i.e., free of aluminum salts. In other words, the antiperspirant composition of the invention is preferably a cosmetic antiperspirant composition which is free of aluminum salt. The expression “aluminum salt” in the sense of the present invention is a general definition of aluminum-containing salts and hence encompasses not only inorganic but also organic salts of aluminum, such as, for example, aluminum chlorohydrates and aluminum hydroxylactates, and also mixed salts of aluminum with other elements, such as zirconium, for example.

In a further embodiment the antiperspirant composition is free of aluminum compounds. In other words, the antiperspirant composition of the invention may additionally, preferably, be an aluminum-free cosmetic antiperspirant composition. The expression “aluminum compounds” in the sense of the present invention is a general definition of compounds of the chemical element aluminum. The expression “aluminum compounds” in the sense of the present invention therefore refers not only to aluminum salts but also, in particular, to aluminum-containing complex compounds, such as aluminum zirconium tetrachlorohydrex-glycin complex, for example.

In another embodiment the antiperspirant composition comprises no additional active antiperspirant ingredient other than the at least one active antiperspirant ingredient provided in accordance with the invention.

In one preferred embodiment the hydroxycarboxylic acid is an α-hydroxycarboxylic acid, preferably a naturally occurring α-hydroxycarboxylic acid.

The hydroxycarboxylic acid is preferably selected from the group consisting of tartaric acid, citric acid, malic acid, gallic acid, and mixtures thereof.

In accordance with the invention, then, it is preferred for the at least one active antiperspirant ingredient to be selected from the group consisting of tartaric acid, citric acid, malic acid, gallic acid, salts thereof, and mixtures thereof. In other words, the at least one active antiperspirant ingredient preferably comprises tartaric acid, citric acid, malic acid, gallic acid, salts thereof and/or mixtures thereof.

In accordance with the invention it may further be preferable if the cosmetic antiperspirant composition comprises as active antiperspirant ingredient/ingredients only tartaric acid, citric acid, malic acid, gallic acid, salts thereof and/or mixtures thereof.

According to one particularly preferred embodiment, the at least one active antiperspirant ingredient comprises tartaric acid or a tartaric salt. The advantages elucidated in accordance with the invention are manifested particularly strongly in the case of tartaric acid and/or a tartaric salt.

In an embodiment which is particularly advantageous from the standpoint of denaturing, the at least one active antiperspirant ingredient has a fraction of 2.5 wt % to 40 wt %, more particularly 3 wt % to 35 wt %, preferably 5 wt % to 30 wt %, more preferably 8 wt % to 25 wt %, most preferably 10 wt % to 20 wt %, based on the total weight of the composition. The invention is also underpinned by the surprising finding that the denaturing properties of the at least one active antiperspirant ingredient provided in accordance with the invention are manifested particularly strongly at the concentrations stated in this paragraph. Another advantageous effect of the active antiperspirant ingredient fractions described in this paragraph is that there is no need for preservatives to be added.

Where the at least one active antiperspirant ingredient comprises tartaric acid or a tartaric acid-containing active antiperspirant ingredient mixture, the tartaric acid may be in a form selected from the group consisting of D-(−)-tartaric acid, L-(+)-tartaric acid, meso-tartaric acid, salts thereof, and mixtures thereof. The tartaric acid may more particularly be in the form of a racemic mixture of D-(−)-tartaric acid (and/or a salt thereof) and L-(+)-tartaric acid (and/or a salt thereof).

Where the active antiperspirant ingredient comprises malic acid or a malic acid-containing active antiperspirant ingredient mixture, the malic acid may be in a form selected from the group consisting of D-(+)-malic acid, L-(−)-malic acid, salts thereof, and mixtures thereof. The malic acid may more particularly be in the form of a racemic mixture of D-(+)-malic acid (and/or a salt thereof) and L-(−)-malic acid (and/or a salt thereof).

In a further embodiment the cosmetic antiperspirant composition further comprises at least one carrier fluid. The expression “carrier fluid” is intended in the sense of the present invention to refer to a preferably liquid carrier material, more particularly a dispersion medium, solvent or suspension medium. The carrier fluid may be selected from the group consisting of ethanol, isopropanol, 2-methylpropan-2-ol, water, and mixtures thereof.

Using ethanol as carrier fluid is especially advantageous, since ethanol, particularly at relatively high concentrations, is strongly denaturing to protein components. By this means it is possible to give an additional boost to the perspiration-inhibiting properties of the composition of the invention. Moreover, particularly at relatively high concentrations, ethanol has an antibacterial effect, meaning that bacteria located on the skin, which break down the sweat and whose excretions are the cause of body odor or perspiration odor, can be killed. In this way an additional boost can be given to the body sweat or body odor control properties of the composition of the invention. Another advantage of ethanol is that there is no need for preservatives to be added.

One suitable ethanolic carrier fluid, for example, is the product available commercially under the tradename Cosmetol (96% form). This product possesses an ethanol fraction of 96 wt %, an isopropanol fraction of 2 wt %, and a 2-methylpropan-2-ol fraction of 2 wt %, based in each case on the total weight of the product.

Another advantage of the use of ethanol as carrier fluid is that together with the at least one active antiperspirant ingredient provided in accordance with the invention, it forms a synergistic active complex whose perspiration-inhibiting effect matches that of a water-based cosmetic formulation containing aluminum salt.

In one preferred embodiment the composition of the invention has an ethanol fraction of 10 wt % to 50 wt %, more particularly 15 wt % to 40 wt %, preferably 20 wt % to 30 wt %, based on the total weight of the composition. The synergistic effect of ethanol in interaction with the at least one active antiperspirant ingredient provided in accordance with the invention is manifested to particularly good effect at concentrations identified in this paragraph.

The cosmetic antiperspirant composition, in a further embodiment, has a water fraction of 10 wt % to 75 wt %, preferably 35 wt % to 50 wt %, based on the total weight of the composition.

In another embodiment the cosmetic antiperspirant composition further comprises Saccharomyces ferment. This is an end product of fermentation by Saccharomyces, and hence is a sugar yeast ferment. A ferment of this kind is available commercially, for example, under the tradename Deosent, DeoPlex Clear H5630. Saccharomyces ferment possesses with particular advantage an odor-masking or odor-reducing effect. This effect arises from enzymatic conversion of particular odorant groups, converting corresponding odorants or fragrances into larger, heavier compounds, which on account of their greater weight are no longer able to evaporate so easily and whose odor is therefore no longer perceived.

The Saccharomyces ferment preferably has a fraction of 0.5 wt % to 5 wt %, more particularly 0.75 wt % to 4 wt %, preferably 1 wt % to 3 wt %, based on the total weight of the composition.

In another embodiment the cosmetic antiperspirant composition further comprises at least one pH regulator. The pH regulator is preferably a compound which is inert toward the active antiperspirant ingredients proposed in accordance with the invention, more particularly toward tartaric acid. The pH regulator may for example be triethanolamine.

The at least one pH regulator preferably has a fraction of 0 wt % to 2.5 wt %, more particularly 0.01 wt % to 2.5 wt %, preferably 2 wt % to 2.5 wt %, based on the total weight of the composition.

In another embodiment the cosmetic antiperspirant composition further comprises at least one complexing agent. The at least one complexing agent preferably comprises polyphosphates. The at least one complexing agent may be selected, for example, from the group consisting of sodium polyphosphate, sodium hexametaphosphate, potassium polyphosphate, calcium polyphosphate, sodium calcium polyphosphate, and mixtures thereof.

The at least one complexing agent preferably has a fraction of 0 wt % to 2.5 wt %, more particularly 0.01 wt % to 2.5 wt %, preferably 0.02 wt % to 0.8 wt %, based on the total weight of the composition.

In another embodiment the cosmetic antiperspirant composition further comprises at least one viscosity regulator and/or film-former. The at least one viscosity regulator and/or film-former may be selected from the group consisting of methylhydroxyethylcellulose, carboxymethylcellulose, methylcellulose, ethylcellulose, hydroxyethylcellulose, hydroxyethylmethylcellulose, hydroxypropylmethylcellulose, homopolymeric polyacrylic acid (so-called carbomers), heteropolymeric polyacrylic acid or copolymers containing acrylate units, guaran, guar gum, hydroxypropyl guar (guar gum, 2-hydroxypropyl ether, depolymerized) such as Esaflor® HDR, carboxymethyl guar, and mixtures thereof

The at least one viscosity regulator and/or film-former preferably has a fraction of 0 wt % to 4 wt %, preferably 0.01 wt % to 2 wt %, more particularly 0.01 wt % to 0.5 wt %, based on the total weight of the composition.

In order to achieve improved penetration of the skin barrier, i.e., improved dermal absorption, the cosmetic antiperspirant composition, in a further embodiment, further comprises at least one surfactant, more particularly at least one emulsifying surfactant.

The at least one surfactant may be selected from the group consisting of polyoxyethylene(20)-sorbitan monolaurate, cocoglucosides such as Plantacare 818, cocamidopropylbetaine, coco-betaines such as Dehyton PK 45 and/or Tego Betaine F50, and mixtures thereof. Polyoxyethylene(20)-sorbitan monolaurate is available commercially under the tradename Polysorbat 20, for example.

The at least one surfactant preferably has a fraction of 0 wt % to 2 wt %, more particularly 0.01 wt % to 2 wt %, preferably 0.2 wt % to 1 wt %, based on the total weight of the composition.

In an embodiment which is advantageous from the standpoint of skincare, the cosmetic antiperspirant composition further comprises at least one skincare substance, more particularly at least one humectant and/or skin moisturizer.

The at least one skincare substance may be selected from the group consisting of amino acids, lactic acid and salts thereof, lactitol, urea, urea derivatives, uric acid, glucosamine, creatinine, cleavage products of collagen, chitosan, chitosan salts/derivatives, polyols and polyol derivatives, such as, for example, glycerol, diglycerol, allantoin, dexpanthenol, triglycerol, glycerol esters such as glyceryl caprylate, ethylene glycol, propylene glycol, butylene glycol, erythritol, 1,2,6-hexanetriol, polyethylene glycols, such as, for example, PEG-4, PEG-6, PEG-7, PEG-8, PEG-9, PEG-10, PEG-12, PEG-14, PEG-16, PEG-18, PEG-20, sugars and sugar derivatives, such as, for example, fructose, glucose, maltose, maltitol, mannitol, inositol, sorbitol, sorbitylsilanediol, sucrose, trehalose, xylose, xylitol, glycuronic acid and salts thereof, and also mixtures thereof

The at least one skincare substance is preferably selected from the group consisting of urea, propane-1,2-diol, glycerol, diglycerol, allantoin, dexpanthenol, and mixtures thereof.

The at least one skincare substance preferably has a fraction of 0 wt % to 20 wt %, more particularly 0.01 wt % to 20 wt %, preferably 6 wt % to 17 wt %, based on the total weight of the composition.

The cosmetic antiperspirant composition may for example have a urea fraction of 0 wt % to 5 wt %, more particularly 0.01 wt % to 5 wt %, preferably 1 wt % to 3 wt %, based on the total weight of the composition.

Furthermore, the cosmetic antiperspirant composition may have a propane-1,2-diol fraction of 0 wt % to 10 wt %, more particularly 0.01 wt % to 10 wt %, preferably 2 wt % to 6 wt %, based on the total weight of the composition.

Furthermore, the cosmetic antiperspirant composition may have a glycerol fraction of 0 wt % to 10 wt %, more particularly 0.01 wt % to 10 wt %, preferably 2 wt % to 6 wt %, based on the total weight of the composition.

Furthermore, the cosmetic antiperspirant composition may have an allantoin fraction of 0 wt % to 0.5 wt %, more particularly 0.01 wt % to 0.5 wt %, preferably 0.3 wt % to 0.5 wt %, based on the total weight of the composition.

Furthermore, the cosmetic antiperspirant composition may have a dexpanthenol fraction of 0 wt % to 5 wt %, more particularly 0.01 wt % to 5 wt %, preferably 0.5 wt % to 1 wt %, based on the total weight of the composition.

In an embodiment which is advantageous from the standpoint of odor neutralization, the cosmetic antiperspirant composition further comprises at least one active deodorant ingredient. The at least one active deodorant ingredient may be selected from the group consisting of fragrances and odorants, perfume (liquid mixture of alcohol, especially ethanol, and odorants or fragrances), odor absorbers or odor extinguishers, active antimicrobial ingredients, enzyme inhibitors, and mixtures thereof.

For example, the cosmetic antiperspirant composition may further comprise at least one fragrance or odorant. As far as the at least one odorant or fragrance is concerned, there are in principle no restrictions at all, provided the odorant or fragrance in question is one which tolerates a slightly acidic environment. The at least one fragrance or odorant may for example be menthol.

The cosmetic antiperspirant composition preferably has a fragrance and/or odorant fraction of 0 wt % to 0.2 wt %, more particularly 0.01 wt % to 0.2 wt %, preferably 0.05 wt % to 0.1 wt %, based on the total weight of the composition.

Suitable odor absorbers and odor extinguishers are silicates, especially phyllosilicates, such as, for example, montmorillonite, kaolinite, beidellite, saponite, bentonite, zeolites, cyclodextrins, and mixtures thereof

Suitable active antimicrobial ingredients may be selected from the group consisting of triclosan, chlorhexidine, chlorhexidine gluconate, bromochlorophene, dichlorophene, chlorothymol, chloroxylenol, hexachlorophene, ammonium phenolsulfonate, benzalkonium halides, benzalkonium cetyl phosphate, benzalkonium saccharinate, benzethonium chloride, cetylpyridinium chloride, laurylpyridinium chloride, laurylisoquinolinium bromide, methylbenzethonium chloride, terpene alcohols, such as, for example, farnesol, chlorophyllin-copper complexes, carboxylic esters of mono-, di- and/or triglycerol, such as, for example, glycerol monolaurate and/or diglycerol monocaprate, fatty acids, plant extracts, such as green tea and/or lime blossom oil constituent, for example, and mixtures thereof

In a further embodiment the cosmetic antiperspirant composition further comprises at least one preservative. The at least one preservative may be selected from the group consisting of benzoic acid and derivatives thereof, propionic acid and derivatives thereof, salicylic acid and derivatives thereof, alcohols and derivatives thereof, guaiacol and derivatives thereof, hydantoin and derivatives thereof, ferulic acid and derivatives thereof, sorbic acid and derivatives thereof, and mixtures thereof.

Provision may be made in accordance with the invention for the cosmetic antiperspirant composition to have a preservative fraction of 0.01 wt % to 1 wt %, preferably 0.1 wt % to 0.5 wt %, based on the total weight of the composition.

In an alternative embodiment the cosmetic antiperspirant composition is free of a preservative. As already mentioned, the addition of preservatives may be unnecessary because of the use of at least one of the proposed active antiperspirant ingredients and/or the use of ethanol as carrier fluid.

In another embodiment the cosmetic antiperspirant composition further comprises at least one antioxidant. The at least one antioxidant may be selected from the group consisting of amino acids, such as, for example, glycine, histidine, tyrosine, tryptophan, amino acid derivatives, imidazole, imidazole derivatives, such as, for example, urocanic acid, carotenoids, carotenes, such as, for example, α-carotene, β-carotene and/or lycopene, carotenoid derivatives, carotene derivatives, lipoic acid and derivatives thereof, such as, for example, dihydrolipoic acid, thio compounds, humic acids, bile acid, bile extracts, flavonoids, catechins, bilirubin, biliverdin, unsaturated fatty acids and derivatives thereof, such as, for example, γ-linolenic acid, linoleic acid, arachidonic acid, oleic acid, folic acid and derivatives thereof, hydroquinone and its derivatives, ubiquinone and ubiquinol and also derivatives thereof, vitamin C and its derivatives, tocopherols and derivatives thereof, and mixtures thereof.

Provision may be made in accordance with the invention for the cosmetic antiperspirant composition to have an antioxidant fraction of 0.01 wt % to 1 wt %, preferably 0.1 wt % to 0.5 wt %, based on the total weight of the composition.

In another embodiment, the cosmetic antiperspirant composition further comprises at least one zinc compound, more particularly at least one zinc salt, such as zinc oxide, for example.

In a further embodiment the cosmetic antiperspirant composition further comprises at least one antiadhesive compound. The at least one antiadhesive compound may comprise, in particular, silicone-based compounds, such as cyclopentasiloxanes, for example.

In one preferred embodiment the composition contains to 100 wt % the following ingredients:
5 wt % to 30 wt %, preferably 10 wt % to 20 wt %, of the at least one active antiperspirant ingredient,
10 wt % to 50 wt %, preferably 20 wt % to 30 wt %, of ethanol,
0.5 wt % to 5 wt %, preferably 1 wt % to 3 wt %, of Saccharomyces ferment,
0 wt % to 2.5 wt %, preferably 2 wt % to 2.5 wt %, of at least one pH regulator,
0 wt % to 2.5 wt %, preferably 0.2 wt % to 0.8 wt %, of at least one complexing agent,
0 wt % to 4 wt %, preferably 0.01 wt % to 2 wt %, of at least one viscosity regulator and/or film-former,
0 wt % to 2 wt %, preferably 0.2 wt % to 1 wt %, of at least one surfactant,
0 wt % to 30.5 wt %, preferably 5.5 wt % to 16.5 wt %, of at least one skincare substance,
0 wt % to 0.2 wt %, preferably 0.05 wt % to 0.1 wt %, of at least one fragrance, and
10 wt % to 75 wt %, preferably 35 wt % to 50 wt % of water.

In one particularly preferred embodiment the composition contains to 100 wt % the following ingredients:
5 wt % to 30 wt %, preferably 10 wt % to 20 wt %, of tartaric acid or tartaric salt,
10 wt % to 50 wt %, preferably 20 wt % to 30 wt %, of ethanol,
0.5 wt % to 5 wt %, preferably 1 wt % to 3 wt %, of Saccharomyces ferment,
0 wt % to 2.5 wt %, preferably 2 wt % to 2.5 wt %, of triethanolamine,
0 wt % to 2.5 wt %, preferably 0.2 wt % to 0.8 wt %, of sodium polyphosphate,
0 wt % to 4 wt %, preferably 0.01 wt % to 2 wt %, of methylhydroxyethylcellulose or hydroxypropyl guar,
0 wt % to 2 wt %, preferably 0.2 wt % to 1 wt %, of polyoxyethylene(20)-sorbitan monolaurate,
0 wt % to 5 wt %, preferably 1 wt % to 3 wt %, of urea,
0 wt % to 10 wt %, preferably 2 wt % to 6 wt %, of propane-1,2-diol,
0 wt % to 10 wt %, preferably 2 wt % to 6 wt %, of glycerol,
0 wt % to 0.5 wt %, preferably 0.3 wt % to 0.5 wt %, of allantoin,
0 wt % to 5 wt %, preferably 0.5 wt % to 1 wt %, of dexpanthenol,
0 wt % to 0.2 wt %, preferably 0.05 wt % to 0.1 wt %, of menthol or perfume, and
10 wt % to 75 wt %, preferably 35 wt % to 50 wt % of water.

In a further embodiment the cosmetic antiperspirant composition consists of at least one of the inventively provided active antiperspirant ingredients and also of at least one further ingredient, which is preferably selected from the group consisting of carrier fluids, pH regulators, complexing agents, viscosity regulators and/or film-formers, surfactants, skincare substances, preservatives, antioxidants, zinc compounds, antiadhesive compounds, and mixtures thereof. With regard to the at least one inventively provided active antiperspirant ingredient and to the ingredients, reference is made in full to the description so far.

In accordance with the invention, for example, provision may be made for the cosmetic antiperspirant composition to consist of at least one of the inventively provided active antiperspirant ingredients, at least one carrier fluid, at least one pH regulator, at least one complexing agent, at least one viscosity regulator and/or film-former, at least one surfactant, at least one skincare substance, at least one fragrance and/or odorant, and optionally at least one antiadhesive compound and/or at least one zinc compound. With regard to the at least one inventively provided active antiperspirant ingredient and to the ingredients, reference is likewise made in full to the description so far.

In another embodiment the cosmetic antiperspirant composition is formulated as a solution, suspension, gel, more particularly hydrogel, cream, paste, salve, roll-on composition, stick, spray sud, pump spray or, together with a propellant, as an aerosol spray.

In other words, it is preferred in accordance with the invention if the antiperspirant composition is in the form of a solution, a suspension, a gel, more particularly hydrogel, a cream, a paste, a salve, a roll-on composition, a stick, a spray sud, a pump spray or, together with a propellant, in the form of an aerosol spray.

According to a second aspect, the invention relates to a cosmetic product which comprises an antiperspirant composition as per the first invention aspect or which consists of such a composition.

The cosmetic product is preferably a cosmetic gel such as cosmetic hydrogel, a cosmetic cream, a cosmetic paste, a cosmetic salve, a cosmetic roll-on product, a cosmetic stick, a cosmetic spray sud, a cosmetic pump spray or a cosmetic aerosol spray. In the case of a cosmetic aerosol spray configuration, the cosmetic product may comprise a suitable propellant as well as an antiperspirant composition as per the first invention aspect.

For further features and advantages of the cosmetic product, especially of the antiperspirant composition, reference is made in full to the observations given in the context of the first invention aspect.

According to a third aspect, the invention relates to a hydroxycarboxylic acid and/or a salt thereof for use in the production of a cosmetic antiperspirant composition free of aluminum salt, more particularly for use in the production of a cosmetic antiperspirant composition which is free of aluminum compounds.

The hydroxycarboxylic acid is preferably an α-hydroxycarboxylic acid, more particularly a naturally occurring α-hydroxycarboxylic acid.

The hydroxycarboxylic acid is preferably selected from the group consisting of tartaric acid, citric acid, malic acid, gallic acid, and mixtures thereof.

For further features and advantages of the hydroxycarboxylic acid, the salt of the hydroxycarboxylic acid, and the cosmetic antiperspirant composition, reference is made in full to the observations given in the context of the invention aspects so far.

Further features and advantages of the invention are apparent from the description hereinafter of preferred embodiments in the form of examples, and also from the dependent claims. The preferred embodiments serve merely for better understanding of the invention, without confining the invention to these embodiments.

### EXAMPLES SECTION

1. Production of Antiperspirant Compositions

1.1 Production of a Perspiration-inhibiting Composition for Use as Pump Spray with Long-lasting Action

A stirring vessel was charged first of all with 225.625 g of heated water (50° C.), 0.500 g of sodium hexametaphosphate, 55.000 g of tartaric acid, and 12.500 g of triethanolamine (99% form). The stirring vessel was then stirred for a period of 3 minutes.

Subsequently 15.000 g of urea, 2.500 g of allantoin, 30.000 g of propylene glycol (1.2 USP), 20.000 g of glycerol (99% form), and 3.000 g of Sorbital T 20 P were added. The stirring vessel was left with stirring for a further period of 6 minutes. Thereafter 0.375 g of menthol (crystalline), 125.000 g of Cosmetol (96% form), 7.500 g of Deosent/DeoPlex Clear, and 3.000 g of dexpanthenol were added. The stirring vessel was again left with stirring for a period of 6 minutes.

This gave a colorless, clear composition of low viscosity with an alcoholic odor, which contained the above ingredients in the following fractions:

45.12 wt % water

0.10 wt % sodium hexametaphosphate

11.00 wt % tartaric acid

2.50 wt % triethanolamine (99% form)

3.00 wt % urea

0.50 wt % allantoin

6.00 wt % propylene glycol (1.2 USP)

4.00 wt % glycerol (99% form)

0.60 wt % Sorbital (T 20 P)

0.08 wt % menthol (crystalline)

25 wt % Cosmetol (96% form)

1.50 wt % Deosent/DeoPlex Clear

0.60 wt % dexpanthenol.

1.2 Production of a Further Perspiration-inhibiting Composition for Use as Pump Spray with 24-hour Action

A stirring vessel was charged first of all with 253.625 g of heated water (50° C.), 0.500 g of sodium hexametaphosphate, 27.500 g of tartaric acid, and 12.500 g of triethanolamine (99% form). The stirring vessel was then stirred for a period of 3 minutes.

Subsequently 15.000 g of urea, 2.000 g of allantoin, 30.000 g of propylene glycol (1.2 USP), 20.000 g of glycerol (99% form), and 3.000 g of Sorbital T 20 P were added. The stirring vessel was left with stirring for a period of further 6 minutes.

Thereafter 0.375 g of menthol (crystalline), 125.000 g of Cosmetol (96% form), 7.5000 g of Deosent/DeoPlex Clear, and 3.000 g of dexpanthenol were added. The stirring vessel was again left with stirring for a period of 6 minutes.

This gave a colorless, clear composition of medium viscosity with an alcoholic odor, which contained the above ingredients in the following fractions:

50.72 wt % water

0.10 wt % sodium hexametaphosphate

5.50 wt % tartaric acid

2.50 wt % triethanolamine (99% form)

3.00 wt % urea

0.40 wt % allantoin

6.00 wt % propylene glycol (1.2 USP)

4.00 wt % glycerol (99% form)

0.60 wt % Sorbital T 20 P

0.08 wt % menthol (crystalline)

25.00 wt % Cosmetol (96% form)

1.50 wt % Deosent/DeoPlex Clear

0.60 wt % dexpanthenol

1.3 Production of a Perspiration-inhibiting Composition as Roll-on with Long-lasting Action

A stirring vessel was charged first of all with 219.125 g of heated water (50° C.), 1.500 g of sodium hexametaphosphate, 55.000 g of tartaric acid, and 12.500 g of triethanolamine (99% form). The stirring vessel was then stirred for a period of 3 minutes. The resulting solution was then filtered in a separate vessel, after which it was returned to the stirring vessel.

Subsequently 15.000 g of urea, 2.500 g of allantoin, 45.000 g of dicylercol and 3.000 g of Sorbital T 20 P were added. The stirring vessel was left with stirring for a period of 6 minutes, with 0.500 g of Tylose H 4000 NG4 being added during the stirring process.

Thereafter 0.375 g of menthol (crystalline), 125.000 g of Cosmetol (96% form), 10.000 g of Xiameter (PMX-245), 7.500 g of Deosent/DeoPlex Clear, and 3.000 g of dexpanthenol were added. The stirring vessel was again left with stirring for a period of 6 minutes.

This gave a colorless, viscous composition with a clear appearance and an odor of alcohol, which contained the above ingredients in the following fractions:

43.82 wt % water

0.30 wt % sodium hexametaphosphate

11.00 wt % tartaric acid

2.50 wt % triethanolamine (99% form)

3.00 wt % urea

0.50 wt % allantoin

9.00 wt % diglycerol

0.60 wt % Sorbital T 20 P

0.10 wt % Tylose H 4000 NG4

0.08 wt % menthol (crystalline)

25.00 wt % Cosmetol (96% form)

2.00 wt % Xiameter PMX-245

1.50 wt % Deosent/DeoPlex Clear

0.60 wt % dexpanthenol.

1.4 Production of a Perspiration-inhibiting Gel with Long-lasting Action

A stirring vessel was charged first of all with 129.625 g of heated water)(50°, 1.500 g of sodium hexametaphosphate, 5.000 g of Esaflor® HDR and 12.500 g of triethanolamine (99% form). The stirring vessel was then left with stirring for a period of 3 minutes.

Subsequently 15.000 g of urea, 2.500 g of allantoin, 40.000 g of glycerol (99% form), 3.000 g of Sorbital T 20 P and 125.000 g of Cosmetol (96% form) were added. The stirring vessel was then left with stirring for a period of 6 minutes.

In a separate vessel, 100.000 g of heated water (50° C.) and 55.000 g of tartaric acid were mixed with one another to give a solution. The solution obtained was then filtered and subsequently introduced into the stirring vessel.

Then 0.375 g of menthol (crystalline), 7.500 g of Deosent/DeoPlex Clear and 3.000 g of dexpanthenol were added to the stirring vessel. Thereafter the stirring vessel was again stirred for a period of 6 minutes.

This gave a colorless, gelatinous composition of minimal turbidity, smelling of alcohol and of high viscosity, which had the above ingredients in the following fractions:

45.92 wt % water

0.30 wt % sodium hexametaphosphate

1.00 wt % Esaflor® HDR

2.50 wt % triethanolamine (99% form)

3.00 wt % urea

0.50 wt % allantoin

8.00 wt % glycerol (99% form)

0.60 wt % Sorbital T 20 P

25.00 wt % Cosmetol (96% form)

11.00 wt % tartaric acid

0.08 wt % menthol (crystalline)

1.50 wt % Deosent/DeoPlex Clear

0.60 wt % dexpanthenol

1.5 Production of a Further Perspiration-inhibiting Composition for use as a Pump Spray with 48-hour Activity Period

A stirring vessel was charged first of all with 194.25 g of heated water (50° C.), 35.00 g of tartaric acid and 12.50 g of triethanolamine (99% form). Thereafter the stirring vessel was left with stirring for a period of 3 minutes.

Subsequently 2.00 g of allantoin, 1.50 g of urea, 45.00 g of glycerol (99% form) and 10.00 g of glyceryl caprylate were added. The stirring vessel was left with stirring for 6 minutes more. Thereafter 3.75 g of Deosent or Deoplex Clear, 7.50 g of dexpanthenol and 188.50 g of Cosmetol (96% form) were added. The stirring vessel was again left with stirring for a period of 6 minutes.

A colorless solution of low viscosity was obtained that had the above ingredients in the following fractions:

38.85 wt % water

7.00 wt % tartaric acid

2.50 wt % triethanolamine (99% form)

0.40 wt % allantoin

0.30 wt % urea

9.00 wt % glycerol (99% form)

2.00 wt % glyceryl caprylate

0.75 wt % Deosent/Deoplex Clear

1.50 wt % dexpanthenol

37.70 wt % Cosmetol (96% form)

1.6 Production of a Further Perspiration-inhibiting Roll-on Composition with 48-hour Activity Period

A stirring vessel was charged first of all with 193.50 g of heated water (50° C.), 7.50 g of Esaflor HDR, 35.00 g of tartaric acid and 12.50 g of triethanolamine (99% form). Thereafter the stirring vessel was left with stirring for a period of 3 minutes. The resulting solution was then filtered into a separate vessel and subsequently returned to the stirring vessel.

Subsequently 2.00 g of allantoin, 1.50 g of urea, 45.00 g of glycerol (99% form) and 10.00 g of glyceryl caprylate were added. The stirring vessel was left with stirring for a period of 6 minutes. Thereafter 3.75 g of Deosent/Deoplex Clear, 7.50 g of dexpanthenol and 188.50 g of Cosmetol (96% form) were added. The stirring vessel was again left with stirring for a period of 6 minutes.

A colorless solution of low viscosity was obtained that had the above ingredients in the following fractions:

38.70 wt % water

0.15 wt % Esaflor HDR

7.00 wt % tartaric acid

2.50 wt % triethanolamine (99% form)

0.40 wt % allantoin

0.30 wt % urea

9.00 wt % glycerol (99% form)

2.00 wt % glyceryl caprylate

0.75 wt % Deosent/Deoplex Clear

1.50 wt % dexpanthenol

37.70 wt % Cosmetol (96% form)

1.7 Production of a Further Perspiration-inhibiting, Fragrance-containing Composition for Use as a Pump Spray with 24-hour Activity Period

A stirring vessel was charged first of all with 242.50 g of heated water (50° C.) and 18.75 g of tartaric acid. Thereafter the stirring vessel was left with stirring for a period of 3 minutes.

Subsequently 2.50 g of allantoin, 1.00 g of urea, 45.00 g of glycerol (99% form) and 6.25 g of glyceryl caprylate were added. Thereafter the stirring vessel was left with stirring for a period of 6 minutes.

Subsequently 8.25 g of dexpanthenol, 175.00 g of Cosmetol (96% form) and 0.75 g of a perfume composition were added. The stirring vessel was again left with stirring for a period of 6 minutes.

A colorless solution of low viscosity was obtained that had the above ingredients in the following fractions:

48.50 wt % water

3.75 wt % tartaric acid

0.50 wt % allantoin

0.20 wt % urea

9.00 wt % glycerol (99% form)

1.25 wt % glyceryl caprylate

1.65 wt % dexpanthenol

35.00 wt % Cosmetol (96% form)

0.15 wt % perfume composition

1.8 Production of a further perspiration-inhibiting, fragrance-containing composition for use as a roll-on with 24-hour activity period

A stirring vessel was charged first of all with 243.00 g of heated water (50° C.), 0.75 g of Esaflor HDR and 20.00 g of tartaric acid. Thereafter the stirring vessel was left with stirring for a period of 3 minutes.

Subsequently 2.50 g of allantoin, 1.00 g of urea, 42.50 g of glycerol (99% form) and 6.25 g of glyceryl caprylate were added. Thereafter the stirring vessel was left with stirring for a period of 6 minutes.

Subsequently 8.25 g of dexpanthenol, 175.00 g of Cosmetol (96% form) and 0.75 g of a perfume composition were added. The stirring vessel was again left with stirring for a period of 6 minutes.

A colorless solution of medium viscosity was obtained that had the above ingredients in the following fractions:

48.60 wt % water

0.15 wt % Esaflor HDR

4.00 wt % tartaric acid

0.50 wt % allantoin

0.20 wt % urea

8.50 wt % glycerol (99% form)

1.25 wt % glyceryl caprylate

1.65 wt % dexpanthenol

35.00 wt % Cosmetol (96% form)

0.15 wt % perfume composition
2. Testing of the Compositions Produced in Examples 1.1 to 1.8 for their Perspiration-inhibiting Activity

2.1 Person with Severe Underarm Perspiration

The compositions produced in examples 1.1-1.8 were each tested on persons of different sex aged from 15 to 48 years and suffering from generally severe underarm perspiration. Application of the compositions to the axillary areas affected led in each case to a significant reduction in the amount of perspiration and also to the absence of perspiration odor. In the case of the compositions produced in examples 1.1, 1.3 and 1.4, the desired effect lasted over a period of up to 48 hours. In the case of the compositions produced in examples 1.2, 1.7 and 1.8, the desired effect lasted over a period of up to 18 hours. In the case of the compositions produced in examples 1.5 and 1.6, the desired effect lasted over a period of more than 24 hours.

2.2 Person with Normal Underarm Perspiration

The compositions produced in examples 1.1-1.8 were each tested on persons of different sex aged from 16 to 49 years and having normal axillary perspiration. Application of the compositions to the axillary areas affected led in each case to a significant reduction in the amount of perspiration and also to the absence of perspiration odor. In the case of the compositions produced in examples 1.1, 1.3 and 1.4, the desired effect lasted over a period of more than 48 hours. In the case of the compositions produced in examples 1.2, 1.7 and 1.8, the desired effect lasted over a period of up to 24 hours. In the case of the compositions produced in examples 1.5 and 1.6, the desired effect lasted over a period of up to 48 hours.

1-15. (canceled)

16. A cosmetic antiperspirant composition free of aluminum salt and comprising at least one active antiperspirant ingredient selected from the group consisting of hydroxycarboxylic acid, hydroxycarboxylic salt, and mixtures thereof.

17. The antiperspirant composition as claimed in claim 16, characterized in that the hydroxycarboxylic acid is an α-hydroxycarboxylic acid, more particularly a naturally occurring α-hydroxycarboxylic acid.

18. The antiperspirant composition as claimed in claim 16, characterized in that the hydroxycarboxylic acid is selected from the group consisting of tartaric acid, citric acid, malic acid, gallic acid, and mixtures thereof

19. The antiperspirant composition as claimed in claim 18, characterized in that the at least one active antiperspirant ingredient is tartaric acid or a tartaric salt.

20. The antiperspirant composition as claimed in claim 16, characterized in that the at least one active antiperspirant ingredient has a fraction of 2.5 wt % to 40 wt %, preferably 5 wt % to 30 wt %, more preferably 8 wt % to 25 wt %, based on the total weight of the composition.

21. The antiperspirant composition as claimed in claim 16, characterized in that the composition further comprises at least one carrier fluid selected from the group consisting of ethanol, isopropanol, 2-methylpropan-2-ol, water, and mixtures thereof.

22. The antiperspirant composition as claimed in claim 16, characterized in that the composition has an ethanol fraction of 10 wt % to 50 wt %, more particularly 15 wt % to 40 wt %, preferably 20 wt % to 30 wt %, based on the total weight of the composition.

23. The antiperspirant composition as claimed in claim 16, characterized in that the composition has a water fraction of 10 wt % to 75 wt %, preferably 35 wt % to 50 wt %, based on the total weight of the composition.

24. The antiperspirant composition as claimed in claim 16, characterized in that the composition further comprises a Saccharomyces ferment.

25. The antiperspirant composition as claimed in claim 24, characterized in that the ferment has a fraction of 0.5 wt % to 5 wt %, more particularly 0.75 wt % to 4 wt %, preferably 1 wt % to 3 wt %, based on the total weight of the composition.

26. The antiperspirant composition as claimed in claim 18, characterized in that it contains to 100 wt % the following ingredients:
5 wt % to 30 wt % of the at least one active antiperspirant ingredient,
10 wt % to 50 wt % of ethanol,
0.5 wt % to 5 wt % of Saccharomyces ferment,
0 wt % to 2.5 wt % of at least one pH regulator,
0 wt % to 2.5 wt % of at least one complexing agent,
0 wt % to 2.0 wt % of at least one viscosity regulator and/or film-former,
0 wt % to 2 wt % of at least one surfactant,
0 wt % to 30.5 wt % of at least one skincare substance,
0 wt % to 0.2 wt % of at least one fragrance, and
10 wt % to 75 wt % of water.

27. The antiperspirant composition as claimed in claim 16, characterized in that the composition is formulated as a solution, suspension, gel, more particularly hydrogel, cream, paste, salve, roll-on composition, stick, spray sud, pump spray or, in combination with a propellant, as an aerosol spray.

28. A cosmetic product comprising or consisting of a composition as claimed in claims 26.

29. A hydroxycarboxylic acid and/or hydroxycarboxylic salt for use in the production of a cosmetic antiperspirant composition free of aluminum salt.
